(19) United States
(12) Patent Application Publication
(10) Pub. No.: US 2003/0175149 A1
(43) Pub. Date: Sep. 18, 2003

(54) Renewable, modifiable, membrane gas exchanger

(76) Inventors: Bruce Searles, Syracuse, NY (US); Edward M. Darling, Baldwinsville, NY (US); Xiao-Mang You, Dewitt, NY (US)

Correspondence Address: HESLIN ROTHENBERG FARLEY & MESITI PC, 5 COLUMBIA CIRCLE, ALBANY, NY, 12203, US

(21) Appl. No.: 10/100,692

(22) Filed: Mar. 18, 2002

(51) Int. Cl. A61M 1/14; A61M 37/00; A61M 1/34

(52) U.S. Cl. 422/44; 422/45; 604/6.14

(57) ABSTRACT

An oxygenator capable of a small priming volume includes a housing defining an interior cavity. The interior cavity of the housing is subdivided into at least two chambers by a membrane. One of the chambers is a blood chamber and the other chambers are gas chambers. The oxygenator also includes an inlet tube passing through the housing and piercing the membrane to deliver blood directly into the blood chamber.

### FIELD OF THE INVENTION

This invention relates generally to a membrane gas exchanger, and, more particularly, to an oxygenator usable in an extracorporeal circulatory support system to provide partial or total bypass for small-sized subjects.

### BACKGROUND OF THE INVENTION

During heart surgery, the function of the heart and lungs must be undertaken outside the body of the patient. The lung function is emulated in an oxygenator which must supply fresh oxygen to the blood and remove carbon dioxide. A blood oxygenator must have a sufficient surface area and prime volume to allow proper gas exchange (particularly Co₂ and O₂).

Prime volume is the volume of blood that is pumped through an extracorporeal circulatory support system to “prime” it. Typically, prior to the initiation of surgery, the total internal volume of the extracorporeal circuit, which includes an oxygenator, heat exchanger, cardioplegia line, ventricular vent line, and other components, must be primed. Priming flushes out any extraneous gas from the extracorporeal circuit prior to the introduction of blood. The larger the priming volume, the greater the amount of priming solution present in the circuit which mixes with the patient's blood.

However, the mixing of blood and priming solution causes hemodilution. Hemodilution is disadvantageous and undesirable because the relative concentration of red blood cells must be maintained during the operation in order to minimize adverse effects to the patient.

In order to reduce the deleterious effects of hemodilution, donor blood may be used. However, use of donor blood is undesirable because, while it reduces the disadvantages associated with hemodilution, donor blood presents complications such as compatibility and the potential transmission of disease. Alternatively, hemoconcentrators may be used to counter the effects of hemodilution. However, such devices add an additional cost to the procedure thus increasing an already expensive operation.

Typically, the prime volume of the total extracorporeal circuit ranges from one to two and a half liters and is intended for an older, larger human. Of that volume, the prime liquid in most commercially available oxygenators ranges from 250 mL to 500 mL.

However, the circulating blood volume in small subjects, such as test rodents, organs or limbs, is considerable less than 250 mL, and may even be less than 25 mL. In order to use a circulatory support system equipped with a conventional oxygenator, additional blood must be supplied by, for example, sacrificing additional rodents.

With respect to human limbs or organs which also require a low prime volume, blood would have to be supplied from blood banks. However, blood is typically in short supply and very expensive. Therefore, it is desirable to minimize the prime volume contained within the oxygenator.

While use of oxygenators that are oversized for a particular application results in hemodilution and all of the previously mentioned negative sequela, it also results in increased biochemical activation of biologic solutions (blood/serum) due to the extra surface area of foreign material that the solution comes in contact with. Because commercially available oxygenators are only available in a limited variety of configurations clinicians and researchers are often forced to select an oxygenator which has far more surface area than is necessary to provide adequate gas exchange for their subject. This results in increased immunologic activity of the humoral immune and coagulation systems which have been linked to a variety of morbidities associated with extracorporeal circulation.

In addition, the assembly of conventional oxygenators make it impractical to sterilize the components for re-use or to modify the components after fabrication to accommodate different sized subjects. Therefore, all of the components of current oxygenators are discarded after a single surgery.

### SUMMARY OF THE INVENTION

The shortcomings of the prior art may be alleviated by using an oxygenator in accordance with one or more aspects of the present invention. The oxygenator of the present invention may be used for medical research and clinical medicine. In research, the oxygenator may be part of an extracorporeal circulatory support system in small animal models (e.g. rats) or used with isolated limb or organ perfusion models or used as a gas exchanger for the preparation of solutions with specific partial pressure. In clinical medicine, the oxygenator may be utilized in new surgical techniques in correcting congenital anomalities such as, for example, in-utero fetal open heart surgery.

In one aspect of the invention, there is provided a device for providing gas exchange between separate mediums. The device comprises a housing that defines an interior cavity and a plurality of inlets and outlets for introducing and exiting the mediums. The device further comprises at least one membrane subdividing the interior cavity of the housing into at least two chambers. The at least two chambers each receives a separate medium from the inlets of the housing. The device further comprises an inlet tube in fluid communication with a medium source. The inlet tube passes through one of the inlets of the housing and pierces one of the at least one membrane. The inlet tube delivers one of the mediums to one of the at least two chambers.

In another aspect of the invention, the device functions as an oxygenator in a circulatory support system.

Advantageously, an oxygenator constructed in accordance with the principles of the present invention has certain features which permit the renewal or modification of the components housed in the internal membrane compartment of the oxygenator. One of these features includes a structure which can be easily disassembled and reassembled in order to exchange or renew the internal components or, alternatively, customize these components to conform to different applications. The ability to customize the surface areaof an oxygenation device to exactly meet the needs of the application provides a great advantage to the fields of research and clinical medicine.

Additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 depicts a cross-sectional view of one embodiment of the oxygenator constructed in accordance with an aspect of the present invention; and

FIG. 2 illustrates a schematic of one embodiment of a circulatory support system including an oxygenator made in accordance with an aspect of the present invention.

### DETAILED DESCRITPTION OF THE EMBODIMENTS OF THE INVENTION

Presented herein is a device designed to provide gas exchange between separate mediums across a membrane material. The device may function as an oxygenator in an extracorporeal circulatory support system (e.g cardiopulmonary bypass system) to provide oxygen and carbon dioxide exchange between a gas medium and a blood medium. Although, the device may be used as a gas exchanger for the preparation of solutions in any circulatory support system requiring specific partial pressure requirements.

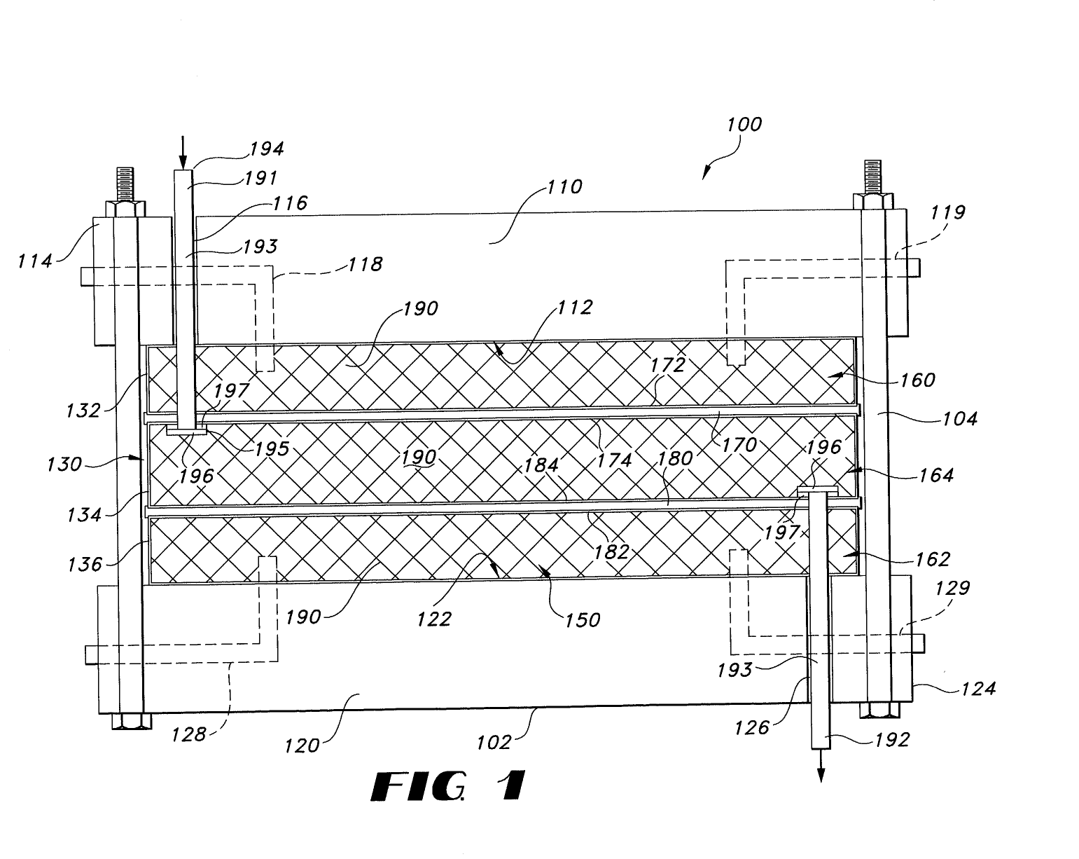

In the illustrative embodiment shown in FIG. 1, oxygenator, generally denoted as 100, comprises a housing 102 including an inlet plate 110, an outlet plate 120, and side walls 130 disposed between inlet plate 110 and outlet plate 120. Inlet plate 110, outlet plate 120 and side walls 130 define an interior cavity 150 which is divided into at least three chambers 160, 162, 164 by a first membrane 170 and a second membrane 180 extending transversely across interior cavity 150 of housing 102. First and second membranes 170, 180, respectively, provide gas exchange (e.g. oxygen and carbon dioxide) between these chambers. Inlet plate 110, outlet plate 120, side walls 130, and first membrane 170 and second membranes 180 are removably secured together by, for example, a plurality of threaded members or bolts 104 along the peripheral edges of and extending between and through inlet plate 110 and outlet plate 120, although other securing means may be used such as, for example, clamps or the like.

Inlet plate 110 includes an inside surface 112 facing first surface 172 of first membrane 170. Upstream end 114 of inlet plate 110 forms blood inlet port 116. As will be described in more detail below, a blood inlet tube 191 may be positioned in fluid communication with tubing connecting, for example, directly to an access vessel of a subject or an output of a pump, and blood chamber 164 defined between first membrane 170 and second membrane 180 within interior cavity 150. Inlet plate 110 also forms a gas inlet port 118 located in proximity to blood inlet port 116 and a gas outlet 119 located near a downstream end of inlet plate 110. Gas inlet 118 and gas outlet 119 are in fluid communication with a gas supply system (not shown).

The gas supply system includes, at least, a source of gas, such as, for example, oxygen, air, and/or carbon dioxide. The gas supply system may also include flow regulators and flow meters to monitor the content and flow of gas in order to prevent, for example, hypoxemia, which is caused by an obstructed oxygen line. Often, when oxygen and air are used, a blender is utilized. An oxygen analyzer may also be incorporated in the gas supply system (after the blender) as well as a micro-filter to filter contaminates from gas. One or more anesthetic vaporizers may also be incorporated in the gas supply line to the oxygenator to supply volatile anesthetic gasses to the gas phase and therefore the blood inducing anesriesia of the subject.

Outlet plate 120 includes an inside surface 122 facing first surface 182 of second membrane 180. Downstream end 124 of outlet plate 120 forms a blood outlet port 126 for exiting, for example, oxygenated blood or other gas enriched solution from blood chamber 164. Blood outlet port 126 may be in fluid communication with tubing connected, for example, directly to a return artery of a subject, and to blood chamber 164 defined between first membrane 170 and second membrane 180 within interior cavity 150. Outlet plate 120 also forms a gas inlet port 128 located at one end of outlet plate 120 and a gas outlet 129 located near a downstream end 124 of outlet plate 120 in fluid communication with the gas supply system.

Side walls 130 may be formed by gaskets 132, 134, 136 disposed between inlet plate 110 and outlet plate 120. Gaskets 132, 134, 136 should be impermeable to fluids and compressible to form a tight seal with inside surfaces 112, 122 of inlet and outlet plates 110, 120, respectively, and adjacent gaskets, in order to prevent fluid from leaking through or around the gaskets. The gaskets should also be biocompatible to prevent the leakage of poisons from the gasket material into the blood and be capable of sustaining heat, sterilization and exposure to gases without crumbling. Suitable materials to form the side walls include, for example, latex, silicon, polypropylene and polycarbonate. In an alternate embodiment, the side walls may be formed by a wall extending from the periphery of one or both of inlet plate 110 and outlet plate 120 towards the other. This wall may be integrally formed with the plates joined to each other in any suitable manner which enables access to the internal components for renewal and/or modification as will be described in more detail below.

Housing 102 of oxygenator 100 may be designed in a variety of shapes, such as, for example rectangular, circular or square. Inlet and outlet plates 110, 120, respectively, may be formed by injection molding from any suitably impervious material, including a thermoplastic material or transparent polymer, such as an acrylic or polycarbonate resin.

Housing 102 of the exemplary oxygenator 100 illustrated in FIG. 1 is configured in three chambers, e.g. two gas chambers 160, 162 and a blood chamber 164. Gas chamber 160 is defined by inside surface 112 of inlet plate 110, the inner surface of gasket 132 and a first side 172 of first membrane 170. Gas chamber 162 is defined by inside surface 122 of outlet plate 120, the inner surface of gasket 134 and first side 182 of second membrane 180. Blood chamber 164 is defined by second side 174 of first membrane 170, second side 184 of second membrane 180 and the inner surface of gasket 136. Blood chamber 164 defines a membrane envelope forming a blood flow path and serves to separate the blood from gas chambers 160, 162.

In an alternate embodiment, housing 102 may be fashioned in two chambers having only one gas chamber and one blood chamber. In this alternate embodiment, the gas chamber is defined by the inside surface of inlet portion 110 and a first side of one membrane and the blood chamber is defined by the inside surface of outlet portion 120 and the second side of a membrane.

First membrane 170 and second membrane 180 extend transversely across interior cavity 150 of housing 102 and are positioned between adjacent gaskets 132 and 134, 134 and 136, respectively, forming side walls 130. Unlike conventional membranes which require a technically complicated and expensive process involving wrapped, fan- folded or rolled membranes, first membrane 170 and second membrane 180 are flat. The use of flat membranes increases the modifiability of the oxygenator at the time of production or application to meet the specific needs of the subject and does not require expensive machinery to produce.

First and second membranes 170, 180 are made of a semipermeable material which permits gas to pass therethough while, at least, retarding the passage of liquid. First and second membranes 170, 180 should resist transferring or absorbing acids, bases and chemicals, should have the ability to be heat-bonded and may have a controlled uniform porosity. In an oxygenator, the pores formed in the membranes permit carbon dioxide from the blood to diffuse from blood chamber 164 into gas chambers 160, 162. Similarly, oxygen from gas chambers 160, 162 permeates through these pores in first and second membranes 170, 180 into the blood flowing through blood chamber 164. Membranes 170, 180 may be cut into any desired shape to correspond to the shape of housing 102. The membranes 170, 180 may be made from, for example, polymer, silicon, polypropylene or polyethylene. One suitable membrane is commercially available from Celgard Inc. of Charlotte, N.C. under the designation Celgard® 2402. This material is made from polypropylene. In alternate embodiments, more than one layer of membrane material may be used to form first membrane 170 or second membrane 180.

A screen spacer 190 may be disposed within each gas chamber 160, 162. Screen spacers 190 maintain the shape of these chambers as blood and/or gas are introduced into oxygenator 100. For example, as blood enters blood chamber 164, pressure within blood chamber 164 increases which may cause first and second membranes 170, 180 to move apart (e.g. widening blood chamber 164). If blood chamber 164 expands too much, the gas exchange between gas chambers 160, 162 may be severely effected or even cut off. With screen spacers 190 in gas chambers 162, 164, the expansion of blood chamber 164 is limited, permitting effective gas exchange between the chambers of interior cavity 150 of housing 102. A screen spacer 190 may also be disposed within blood chamber 164 to maintain the shape of blood chamber 164 during the opposite condition, e.g. when pressure in blood chamber 164 decreases if no blood enters blood chamber 164. Screen spacer may be made from, for example, polypropylene. One suitable screen spacer is commercially available from Small Parts, Inc. of Miami Lakes, Fla. under the designation polypropylene screen cloth.

In an alternate embodiment, instead of using gaskets 132, 134, 136 to form side walls 130, the ends of first and second membranes 170, 180 may be joined together by, for example, ultrasonic welding, bonding, fusion, heat staking, press fitting, heat welding, or other similar means, to inside surfaces 112, 122 of inlet plate 110 and outlet plate 120, respectively, to form gas chambers 160, 162 and blood chamber 164 therebetween. These chambers may be formed similar to an envelope having screen spacers to maintain their shape. In this embodiment, inside surfaces 112, 122 of inlet plate 110 and outlet plate 120, respectively, may be concave to provide additional space to form gas chambers 160, 162 and blood chamber 164.

Blood is delivered directly to blood chamber 164 by an inlet tube 191 which pierces through first membrane 170 and is supported by and may extend out of blood inlet port 116 formed in inlet plate 110. Inlet tube 191 may include a cylindrical tube 193 having a first end 194 extending through inlet plate 110 of housing 102 and a second end 195 extending through first membrane 170. A flange 196 extends radially outward at second end 195 of inlet tube 192 and a gasket 197 (e.g. rubber O-ring) rests on flange 196. Flange 196 and gasket 197 seal the hole created by the piercing of first membrane 170 by compression. In other words, flange 196 presses on gasket 197 which, in turn, presses on second side 174 of first membrane 170 to create a seal between gasket 197 and second side 174 of first membrane 170 which prevents the passage of gas or liquid through the hole formed by the piercing in first membrane 170.

Within blood chamber 164, blood is exposed to gas or air passing through first and second gas chambers 160, 162. Blood exits blood chamber 164 though outlet tube 192 which is supported by blood outlet port 126. Outlet tube 192 pierces through second membrane 180 and extends out blood output port 126 formed in outlet plate 120. Similar to inlet tube 191, outlet tube 192 includes a cylindrical tube 193, a flange 196 at one end 194 of the tube 193 and a gasket 197 resting on flange 196 for compression sealing the hole formed by piercing second membrane 180.

The ideal tubing for inlet tube 191 and outlet tube 192 should minimize blood trauma, minimize prime volume, minimize resistance to blood flow and avoid leaks resulting in the outward flow of blood and aspiration of air. To minimize blood trauma, the inside walls of the tubing should be smooth, non-wettable and nontoxic. By keeping the tubing short, the prime volume, pressure gradient and blood trauma are reduced. Desirable tubing characteristics include transparency, flexibility, kink resistance, hardness to resist collapsing, toughness to resist cracking and rupture, inertness, toleration for heat sterilization, resilience to re-expand after compression and blood compatibility. Some examples of suitable tubing that may be used include medical-grade polyvinyl chloride, silicone, latex rubber and the like, although other materials, such as, for example, stainless steel may be used.

Alternatively, the interfaces between the outer surfaces of the cylindrical tubes 193 of inlet tube 191 and outlet tube 192 and the holes formed in membranes 170, 180 by piercing may be sealed or bonded to prevent leaking of any gas or blood between the chambers 160, 162, 164 of interior 150 at this location.

The internal components of the oxygenator 100, namely first and second membranes 170, 180, inlet tube 191, outlet tube 192, screen spacers 190, and gaskets 132, 134, 136, are intended to be renewable. The remaining components of oxygenator 100, namely inlet plate 110, outlet plate 120 and threaded members 104 may then be cleaned, sterilized and reused. The structure of oxygenator 100 may be easily disassembled by removing threaded members 104 extending between inlet and outlet plate 110, 120. Once disassembled, the internal components can be replaced or renewed, or otherwise modified, at a fraction of the cost of replacing the entire unit.

One method of constructing oxygenator 100 will now be described. First, a plurality of threaded members 104 are inserted around the outer peripheral of inlet plate 110. Gasket 132 is inserted within the space defined by the plurality of threaded members 104 on inside surface 112 of inlet plate 110. A screen spacer 190 is sized to fit against inside surface 112 of inlet plate 110, inside of gasket 132.

Next, first membrane 170 is sized to fit within the space defined by the plurality of threaded members 104 on inside surface 112 of inlet plate 110 and extend beyond gasket 132. At this point, first end 194 of inlet tube 191 is pierced through first membrane 170 and passed through blood inlet port 116 until gasket 197 rests against first membrane 170. Gasket 134 is then inserted on top of first membrane 170 within screen spacer 190 sized to fit inside of gasket 134.

Next, second membrane 180 is also sized to fit within the space defined by the plurality of threaded members 104 and extend beyond gasket 134. Before placing second membrane 180 onto gasket 134, the first end of outlet tube 192 is pierced through second membrane 180 and positioned to pass through blood outlet port 126 in outlet plate 120. Screen spacer 190 is inserted on top of second membrane 180.

Finally, outlet plate 120 is placed over gasket 136 while aligning receiving holes formed in outlet plate 120 for receiving threaded members 104. As threaded members 104 are tightened, gaskets 132, 134, 136 are compressed to form a seal with each other and inside surfaces 112, 122 of inlet plate 110 and outlet plate 120, respectively, and to secure first and second membranes 170 and 180 in place to form gas chambers 160, 162 and blood chamber 164. Gas inlets 118, 119 in inlet plate 110 and gas inlets 128, 129 in outlet plate 120 may be formed prior to assembly of oxygenator 100. Of course, there may be other ways to assemble the entire oxygenator 100, and attach the various components (e.g. bonding first and second membranes to the gaskets forming side wall 130), which are considered part of this invention.

During operation, blood is delivered through inlet tube 191 to blood chamber 164 while oxygen and/or air is delivered to gas chambers 160, 162 from the gas supply system. As the blood flows through blood chamber 164, carbon dioxide from the blood diffuses from blood chamber 164 through first and/or second membrane 170, 180 and into gas chambers 160, 162. At the same time, oxygen from gas chambers 160, 162 permeates through the first and/or second membranes 170, 180 into the blood flowing through blood chamber 164 of housing 102. The oxygenized blood exits blood chamber 164 and housing 102 through outlet tube 192.

In one embodiment, the oxygenator constructed in accordance with the principles of the present invention is capable of oxygenating blood at 100 millimeters per minute with a prime volume of less than 25 milliliters, preferably less than 10 milliliters, and more preferably, less than 5 milliliters. For example, a 450 gram rat requires a prime volume of only 3.0 milliliters. A limb or organ requires a prime volume of only 1.0 milliliters. Of course, the amount of prime volume is dependent on the size of the subject which is attached to the oxygenator and may exceed 25 milliliters, if necessary, to accommodate a larger subject.

In one exemplary application, an oxygenator having a prime volume of about 2.7 milliliters was used in a circulatory support system with a total priming volume of 9.5 milliliters to successfully provide total cardiopulmonary bypass for a 450 gram rat (e.g male Sprague-Dawley) without the need for donor blood or the sacrifice of other rats. With this success, it is envisioned that the oxygenator could be applied, for example, to a 500 to 3000 gram fetus.

Recent advancements in surgical techniques especially in the area of microsurgery and cardiac surgery suggest that there may be a need to oxygenate the developing fetus during surgical intervention while it is still in the womb. Diagnosis and repair of congenital cardiac defects early in the stage of fetal development may produce improved patient outcomes. The repair of these pathologies however will require the support of extracorporeal circulation and oxygenation during the period of surgery. The oxygenators currently available are grossly oversized for this application and cannot be modified by the end user to exactly meet the needs of this patient population.

Monunumental advancements in biotechnology and cell culture in recent years are striving to make a reality the possibility of growing new human organs in the laboratory for transplantation. As this technology continues to advance, there will be a need to provide oxygenation and circulation to in a controlled “BioReactor” environment for developing organs ranging in size from several grams up to 1-2 Kilograms. The present invention represents technology which may be employed in these “BioReactors” which can be customized to the size of the organ and with periodic modifications, “grow” with the organ.

Conventional oxygenators used in clinical medicine or research studies exceed 25 milliliters of prime volume, which is more than the blood volume of, for example, a test rat. In these conventional oxygenators, an additional source of rat blood or, alternatively, a number of rats are required to be sacrificed, in order to provide enough prime volume in the oxygenator and/or circulatory support circuit. With the use of the oxygenator of the present invention, there is no need to sacrifice additional rats to achieve the desired prime volume. The components of the oxygenator of the present invention, such as, for example, membranes, gaskets, and screen spacers, may be easily modified to accommodate the particular prime volume required.

The oxygenator constructed in accordance with the principles of the present invention was evaluated against abbreviated standards for inlet blood conditions as described in the “Guidance for Cardiopulmonary Bypass Oxygenators 510 (k) Submissions” issued on Jan. 17, 2000 and published by the U.S. Department of Health and Human Services Food and Drug Administration, Center for Device and Radiological Health and the Circulatory Support and Prosthetic Devices Branch, Division of Cardiovascular and Respiratory Devices, Office of Device evaluation, which is hereby incorporated herein by reference in its entirety. These standards were developed to provide guidance to industry and FDA staff for the standard evaluation of blood oxygenator (considered an FDA Class II device) As shown in the table below, the oxygenator made in accordance with the principles of the present invention produces clinically acceptable blood outlet conditions when challenged with blood, meeting the FDA standard inlet conditions. For example, the tables shows that the amount of oxygen in the blood increased while the amount of CO₂ in the blood decreased.

| | FDA Standard | Experimental | Experimental |
|---|---|---|---|
| Variable | Inlet | Inlet | Outlet |
| % Saturation of | 65 ± 5 | 61 ± 4 | 100 |
| Hemoglobin with Oxygen | | | |
| Partial | 45 ± 5 | 45 ± 1 | 21 ± 3 |
| Pressure of CO₂ | | | |
| (mmHg) | | | |
| Base Excess | 0 ± 5 | 0 ± 1 | 0 ± 2 |
| Hemoglobin (g/dl) | 12 ± 1 | 12 ± 1 | 12 ± 1 |

The oxygenator constructed in accordance with an aspect of the present invention may be directly connected to an access vessel and a return artery of a subject by tubing. A subject may be, for example, a rodent (e.g. rat), a young infant, an organ, a limb or any other patient or subject requiring a low prime volume oxygenator. The oxygenator may also be connected to one or more components in a circulatory support system, such as, for example, a cardiopulmonary bypass system.

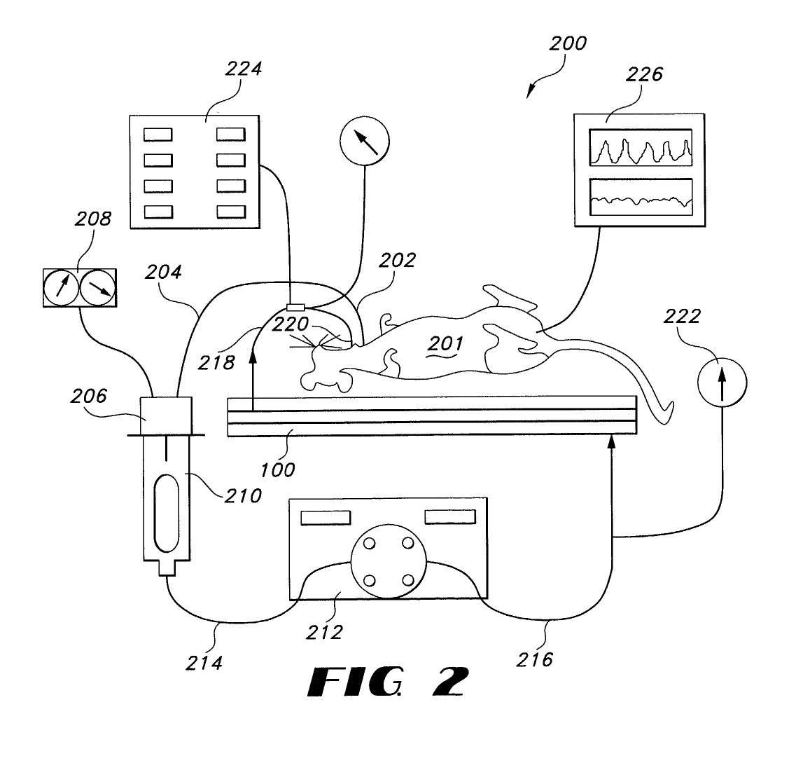

FIG. 2 illustrates one embodiment of a circulatory support system 200 incorporating an oxygenator made in accordance with the principles of the present invention along with a number of other components to aid in supporting a subject (e.g. rodent).

Briefly, system 200 includes an outlet cannula 202 implanted in the internal jugular vein or right ventricle or right atrium of rat 201, although any clinically appropriate access vessel may be used. An outflowing blood flow tube 204 connects outlet cannula 202 and an inlet of a blood reservoir 206 made from, for example, a sealed 30 mL syringe. A vacuum regulator 208 connects to blood reservoir 206 for applying suction (e.g. negative 30 mmHg) to blood reservoir in order to increase venous drainage. Incorporated into reservoir 206 is a heat exchanger 210 connected to a water bath and pump. An outlet of heat exchanger 210 connects to a pump 212 by tubing 214 for generating blood flow. An outgoing blood flow tube 216 connects an outlet of pump 212 to oxygenator 100 (e.g. to inlet tube 191 which delivers the blood through inlet plate 110 and first membrane 170 and into blood chamber 164). The oxygenized blood flows out of oxygenator 100 (e.g. through outlet tube 192) and returns through return blood flow tubing 218 to inlet cannula 220 implanted in the carotid artery, femoral artery or aortic artery of the subject, although any appropriate inlet vessel may be used.

The ideal tubing (e.g. 204, 214, 216, 218) for connecting the various components of the system should minimize blood trauma, minimize prime volume, minimize resistance to blood flow and avoid leaks resulting in the outward flow of blood and aspiration of air. To minimize blood trauma, the inside walls of the tubing should be smooth, non-wettable and nontoxic. By keeping the tubing short, the prime volume, pressure gradient and blood trauma are reduced. Desirable tubing characteristics include transparency, flexibility, kink resistance, hardness to resist collapsing, toughness to resist cracking and rupture, inertness, toleration for heat sterilization, resilience to re-expand after compression and blood compatibility. Some examples of suitable tubing used in cardiopulmonary bypass include medical-grade polyvinyl chloride, silicone, latex rubber and the like.

Reservoir 206 with integral heat exchanger 208 serves as a holding tank or atrium and act as a buffer for fluctuation and imbalances between venous return and arterial flow. Reservoir 206 serves as a high capacity receiving chamber for venous return, facilitating drainage of venous blood. Additionally, while used in a cardiopulmonary bypass system, reservoir 206 is a place to store excess blood when the heart and lungs are exsanguinated. Reservoir 206 may also serve as a gross bubble trap for air that enters the venous line, as the site where blood, fluids or drugs may be added, and as a ready source of blood for transfusion into the subject.

Reservoir 206 also provides time for the perfusionist to act if venous drainage is sharply reduced or stopped, in order to avoid pumping the system dry and risking systemic air embolism. With membrane oxygenators, the reservoir is typically the first component of the extracorporeal circuit, directly receiving the venous drainage as well as the cardiotomy drainage.

Blood passes from reservoir 206 to heat exchanger 208. Heat exchangers are designed to add or remove heat from the blood in order to control body temperature. During its flow in an extracorporeal circuit, blood typically cools and, hence, heat must be added to avoid subject cooling. In addition, the subject's temperature is often deliberately lowered and then needs to be restored to normothermia before discontinuing the bypass. Heat exchangers are usually located in close proximity to the gas exchanging section of the circuit to minimize the risk of releasing micro-bubbles of gas from the blood, which could occur if the blood is warmed after being saturated with gas. A source of hot and cold water, a regulator/blender, and temperature sensors may be added features of heat exchangers. The blood may be circulated through coils of plastic tubing that are placed in an ice bath or, alternatively, a warm water bath.

The blood flow is generated by a pump 212, such as, for example, a roller pump. A roller pump includes a length of tubing, located inside a curved tubing bed between the roller pump housing and the outer perimeter of rollers mounted on the ends of rotating arms. The rotating arms may be, for example, two arms spaced 180 degrees apart or three arms spaced 120 degrees apart. However, other orientations do exist. The roller pump is arranged so that one roller is compressing the tubing at all times. Flow of blood is induced by compressing the tubing which pushes the blood ahead of the moving roller. The tubing behind the rollers recovers its shape, creates a vacuum and draws fluid in behind it. Silastic rubber, latex rubber and polyvinyl chloride tubing may be used in the tubing bed. Polyvinyl chloride, for example, is durable and associated with acceptable rates of hemolysis and will not get stiff during hypothermia and is not subject to release of particulate material. One suitable roller pump is commercially available from Cole-Parmer Instrument Co. under the designation Masterflex. In alternate embodiments, blood flow may be generated by using centrifugal pumps or pulsatile ventricular pumps.

At the output of pump 212, the blood may flow though a flow meter or a pressure gauge or monitor 222 which are placed in line with the tubing attached at the output of pump 212. A flow meter may be used to detect the flow rate of blood being pumped and a pressure gauge detects the hydraulic pressure at which the blood is flowing through the tubing.

Other non-invasive flow-through devices may be positioned throughout the system. These devices may include a variety of temperature, gas and pressure monitors to monitor the flow characteristics in the system and in the body of the subject. For example, a gauge may be used to measure blood gases in the arterial and venous lines. An arterial monitor may provide continuous assessment of arterial oxygenation and permits more rapid and precise control of blood gases. The pressure in the arterial line following pump, but before the oxygenator, may be monitored continuously to detect obstruction in the line, malposition of the arterial cannula, dissection or obstruction of the oxygenator. The temperature of the water supplying the heat exchanger may be monitored to ensure the safe conduct of perfusion. Low-level alarms on the reservoir and a bubble detector on the line are also desirable.

The blood is then pumped through tubing 216 into oxygenator 100. Typically, membrane oxygenators are positioned after the pump because the resistance in most membrane oxygenators requires blood to be pumped through them. As discussed above, oxygenator 100 is attached to a gas supply system.

At the output of oxygenator 100, the oxygenized blood may flow through a monitor 224 which monitors, among things, the flow rate, pressure, temperature or the like. One suitable blood/gas monitoring device for the continuous analysis of the blood gas content is commercially available from Terumo of Tokyo, Japan under the designation CDI 500.

The system may also include a hemodynamic monitor 226 connected to the femoral artery to supervise the pumping characteristics of the heart and the associated blood flows and pressures throughout the cardiovascular system of the subject as a result of the cardiopulmonary bypass system. One suitable hemodynamic monitor is commercially available from ADInstruments Pty Ltd. of Sydney, Australia under the designation PowerLab.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. A device for providing gas exchange between separate mediums; said device comprising:
a housing, said housing defining an interior cavity and a plurality of inlets and outlets configured to introduce and exit the mediums into and out of the interior cavity;
at least one membrane subdividing the interior cavity of said housing into at least two chambers, said at least two chambers each receiving a separate medium from the inlets of said housing;
an inlet tube in fluid communication with a medium source, said inlet tube passing through one of the inlets of said housing and piercing one of the at least one membrane, said inlet tube configured to deliver one of the mediums to one of the at least two chambers.

2. The device of claim 1, wherein said device has a prime volume of less than 5 milliliters.

3. The device of claim 1, wherein said device is an oxygenator.

4. The device of claim 3, wherein one of the at least two chambers in the interior cavity of said housing is a blood chamber, and wherein said inlet tube delivers blood to be oxygenized to the blood chamber.

5. The device of claim 4, wherein the other chambers of the at least two chambers in the interior cavity of said housing are gas chambers, and wherein said medium in the gas chambers includes oxygen.

6. The device of claim 1, wherein said device is part of a circulatory support system.

7. The device of claim 6, wherein the circulatory support system is a extracorporeal circulatory support system.

8. The device of claim 7, wherein the extracorporeal circulatory support system is a cardiopulmonary bypass system.

9. The device of claim 1, wherein said housing includes an inlet plate and an outlet plate, the inlet plate and the outlet plate configured to support internal components therebetween and held together by removable securing means.

10. The device of claim 9, wherein the internal components of said housing, the at least one membrane, and said inlet tube are renewable.

11. The device of claim 10, wherein the internal components include side walls and an outlet tube, the outlet tube being in communication with one of the at least two chambers of the interior cavity of said housing.

12. The device of claim 1, wherein said device includes a first membrane and a second membrane subdividing the interior cavity of the housing into three chambers.

13. The device of claim 12, wherein one of the three chambers is defined between the first membrane and the second membrane, and wherein said inlet tube pierces the first membrane.

14. The device of claim 13 further comprising an outlet tube, said outlet tube passing through one of the outlets of said housing and piercing the second membrane, and said outlet tube configured to exit the medium in the chamber defined between the first and second membranes.

15. The device of claim 13, wherein the chamber defined between the first membrane and the second membrane is a blood chamber.

16. The device of claim 1, wherein the at least one membrane is flat.

17. The device of claim 1, wherein each of the chambers includes a screen spacer.

18. The device of claim 1, wherein said housing includes at least one gas inlet and at least one gas outlet in fluid communication with a gas supply system.

19. The device of claim 1, wherein said inlet tube includes a flange extending radially from an end of said inlet tube pierced through the one of the at least one membrane, and wherein a gasket is positioned between the flange and the one of the at least one membrane to seal any opening exiting in the one of the at least one membrane.

20. A device for providing gas exchange between separate mediums, said device comprising:
a housing, said housing defining an interior cavity and a plurality of inlets and outlets configured to introduce and exit the mediums into and out of the interior cavity, said housing including an inlet plate and an outlet plate;
at least one membrane subdividing the interior of said housing into at least two chambers, said at least two chambers each receiving a separate medium from the inlets of said housing;
an inlet tube in fluid communication with a medium source, said inlet tube passing through one of the inlets of said housing and piercing one of the at least one membrane, said inlet tube configured to deliver one of the mediums to one of the at least two chambers; and
means for securing the inlet plate to the outlet plate with the at least one membrane positioned between the inlet plate and the outlet plate, wherein the securing means is removable so that the at least one membrane and inlet tube may be modified or renewed.

21. A method of assembling a device for providing gas exchange between separate mediums, said method comprising:
providing an inlet plate and an outlet plate, the inlet plate and outlet plate together forming a housing which defines an interior cavity, the inlet plate and outlet plate defining a plurality of inlets and outlets configured to introduce and exit the mediums into and out of the interior cavity;
providing at least one membrane;
providing an inlet tube;
piercing the inlet tube through the at least one membrane;
passing one end of the inlet tube through one of the inlets defined in the inlet plate or outlet plate;
securing the inlet plate to the outlet plate together by a removable securing means with the at least one membrane disposed therebetween, wherein the inlet plate, the at least one membrane and the outlet plate define at least two chambers, the at least two chambers each receiving a separate medium from the inlets of the housing, wherein the other end of the inlet tube extends into one of the at least two chambers; and
renewing the at least one membrane and inlet tube after use of the device.
